(19) United States
(12) Patent Application Publication
(10) Pub. No.: US 2023/0420105 A1
(43) Pub. Date: Dec. 28, 2023

(54) Systems and Methods for Automatic Determination of a Medical Device for Vascular Access

(71) Applicant: Bard Access Systems, Inc., Salt Lake City, UT (US)

(72) Inventors: Anthony K. Misener, Bountiful, UT (US); Steffan Sowards, Salt Lake City, UT (US); William Robert McLaughlin, Bountiful, UT (US)

(21) Appl. No.: 17/849,455

(22) Filed: Jun. 24, 2022

(51) Int. Cl. G16H 20/40; G16H 10/60; G16H 20/10; A61B 5/00

(52) U.S. Cl. CPC G16H 20/40; G16H 10/60; G16H 20/10; A61B 5/7435; A61B 5/7475

(57) ABSTRACT

Disclosed are systems and methods for automatic determination of one or more medical devices for vascular access prior to direct patient assessment. For example, a system can include a console and a display screen optionally integrated into the console. The console can include one or more processors and memory. The memory can include instructions configured to instantiate one or more processes when executed by the one-or-more processors for the automatic determination of the one-or-more medical devices for vascular access in accordance with a plurality of data inputs, various operating parameters, historical data, or a combination thereof. The automatic determination of the one-or-more medical devices can use at least logic, algorithms, machine learning including a machine-learning model trained with the historical data, artificial intelligence, or a combination thereof. The display screen can be configured to display the one-or-more medical devices recommended for vascular access.

### BACKGROUND

Current technologies work to optimize placement of a user-selected medical device for vascular access after ultrasound imaging or the like; however, the user-selected medical device is not always the best medical device for vascular access in view of multifaceted considerations of applicable criteria. That, and time can be saved by at least narrowing a selection of medical devices before even performing the ultrasound imaging.

Disclosed herein are systems and methods for automatic determination of one or more medical devices for vascular access prior to direct patient assessment.

### SUMMARY

Disclosed herein is a system for automatic determination of one or more medical devices for vascular access prior to direct patient assessment. The system includes, in some embodiments, a console. The console includes one or more processors and memory. The memory includes instructions configured to instantiate one or more processes when executed by the one-or-more processors for the automatic determination of the one-or-more medical devices for vascular access in accordance with a plurality of data inputs, various operating parameters, historical data, or a combination thereof. The automatic determination of the one-or-more medical devices uses at least logic, algorithms, machine learning including a machine-learning model trained with the historical data, artificial intelligence, or a combination thereof.

In some embodiments, the plurality of data inputs are automatically pulled into the system.

In some embodiments, the plurality of data inputs include patient condition parameters measured by a measuring device, optionally, operably connected to the system. The patient condition parameters include temperature, blood pressure, blood oxygenation, pH, lactate concentration, glucose levels, or a combination thereof.

In some embodiments, the plurality of data inputs include orders or patient data from an electronic medical-care-facility system. The electronic medical-care-facility system optionally includes access to the patient data via electronic medical records for patients.

In some embodiments, the orders are for specific medical devices, specific medications, or a combination thereof.

In some embodiments, the system further includes a display screen optionally integrated into the console. The display screen is configured to display the one-or-more medical devices recommended for vascular access.

In some embodiments, the various operating parameters include confirmation of clinician training via completion of one or more on-board training modules. The display screen is further configured to display clinician messaging with the confirmation of the clinician training.

In some embodiments, only clinicians having the confirmation of the clinician training are allowed to use the system for the automatic determination of the one-or-more medical devices for vascular access.

In some embodiments, the display screen is further configured to display the clinician messaging with recommendations for escalation. The escalation includes one or more alternative clinicians allowed to use the system for the automatic determination of the one-or-more medical devices for vascular access in view of the one-or-more alternative clinicians having the confirmation of the clinician training.

In some embodiments, the plurality of data inputs are manually input into the system by a clinician using the system.

In some embodiments, the plurality of data inputs include procedure type, one or more clinical rules, clinician experience, one or more clinician preferences, medical-device inventory, one or more orders for specific medical devices, patient data including patient condition or patient location, emergent indication, prior difficulty in blood-vessel access, one or more orders for specific medications, one or more infusion-therapy parameters, one or more imaging parameters if the system includes an imaging modality, dwell time, or a combination thereof.

In some embodiments, the one-or-more clinical rules include purchase length for a medical device, blood-vessel occupancy of the medical device, dwell time for the medical device, or a combination thereof when the medical device is a catheter.

In some embodiments, the one-or-more clinical rules are factored into any automatic recommendations of the one-or-more medical devices for vascular access.

In some embodiments, the one-or-more infusion-therapy parameters include fluid replacement, potassium replacement, heparin, insulin, one or more antibiotics, one or more vesicants, one or more irritants, blood, one or more blood products, pain medication, power-injection parameters, or a combination thereof.

In some embodiments, the one-or-more imaging parameters include at least an intention for power injection while imaging.

In some embodiments, the procedure type includes at least an intention for blood aspiration.

In some embodiments, the prior difficulty in blood-vessel access includes a determination of whether a blood vessel was visible, palpable, torturous, valvular, or a combination thereof.

In some embodiments, the patient condition includes blood pressure, hydration, nutrition, temperature, or a combination thereof.

In some embodiments, the clinician experience includes clinician training for placing the one-or-more medical devices for vascular access or clinician proficiency for placing the one-or-more medical devices for vascular access.

In some embodiments, the system further includes an ultrasound probe operably coupled to the console for ultrasound imaging.

In some embodiments, the system further includes a tip-location sensor configured to rest upon a chest of a patient. The tip-location sensor is configured to locate a tip of a medical device within a vasculature of the patient.

In some embodiments, the system further includes an infusion pump.

In some embodiments, the console is a portable computing device with a display screen integrated therein.

In some embodiments, the system is further for automatic determination of additional procedure-related items for the vascular access with the one-or-more medical devices prior to the direct patient assessment. The additional procedure-related items include gowns, drapes, gloves based upon size, or a combination thereof.

In some embodiments, the automatic determination of the one-or-more medical devices for vascular access is accompanied by a percent likelihood of success, a confidence level, or both, thereby ultimately allowing a clinician to choose a medical device of the one-or-more medical devices.

In some embodiments, the one-or-more processes are further for automatically compiling and analyzing data to identify data trends for reporting internally to a clinic including the system or externally to another care provider.

In some embodiments, the system is configured to train the machine-learning model with the historical data including a clinician-chosen medical device among the one-or-more medical devices recommended for vascular access, clinician feedback on whether the clinician-chosen medical device was successful for vascular access, a tracking record for the clinician-chosen medical device, or a combination thereof.

Also disclosed herein is a method of a system for automatic determination of one or more medical devices for vascular access prior to direct patient assessment. The method includes, in some embodiments, an instantiating step. The instantiating step includes executing instructions in memory of a console by one or more processors of the console, thereby instantiating one or more processes for the automatic determination of the one-or-more medical devices for vascular access in accordance with a plurality of data inputs, various operating parameters, historical data, or a combination thereof. The automatic determination of the one-or-more medical devices uses at least logic, algorithms, machine learning including a machine-learning model trained with the historical data, artificial intelligence, or a combination thereof.

In some embodiments, the method further includes a displaying step. The displaying step includes displaying on a display screen optionally integrated into the console the one-or-more medical devices recommended for vascular access.

These and other features of the concepts provided herein will become more apparent to those of skill in the art in view of the accompanying drawings and following description, which describe particular embodiments of such concepts in greater detail.

### DRAWINGS

FIG. 1 illustrates a system for automatic determination of one or more medical devices for vascular access in accordance with some embodiments.

FIG. 2 illustrates another system for the automatic determination of the one-or-more medical devices for vascular access in accordance with some embodiments.

FIG. 3 illustrates a block diagram of the system of FIG. 1 or 2 in accordance with some embodiments.

FIG. 4 illustrates training one or more machine-learning models (“MLMs”) with historical data of the system in accordance with some embodiments.

FIG. 5 illustrates a flow chart including a plurality of data inputs useful to the system of FIG. 1 or 2 for the automatic determination of the one-or-more medical devices for vascular access in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “logic,” logic refers to hardware, software, or firmware configured to perform one or more functions. As hardware, logic can refer to circuitry having data-processing or storage functionality. Examples of such circuitry include, but are not limited to, a hardware processor (e.g., a microprocessor, one or more processor cores, a digital-signal processor, a programmable gate array [“PGA”], a microcontroller, an application specific integrated circuit [“ASIC”], etc.), semiconductor memory, or the like. As software, logic can refer to one or more processes, one or more instances, Application Programming Interface(s) (API), subroutine(s), function(s), applet(s), servlet(s), routine(s), source code, object code, shared or dynamic link libraries (dll), or even one or more instructions. Such software can be stored in any type of a suitable non-transitory storage medium or transitory storage medium (e.g., electrical signals, optical signals, acoustical signals, or some other form of propagated signals). Examples of a non-transitory storage medium include, but are not limited to, a programmable circuit; a non-persistent storage medium such as volatile memory (e.g., any type of random-access memory [“RAM”]); a persistent storage medium such as non-volatile memory (e.g., read-only memory [“ROM”], power-backed RAM, flash memory, phase-change memory, etc.), a solid-state drive, a hard-disk drive, an optical-disc drive, or a portable memory device. As firmware, logic can be stored in persistent storage.

As used herein, a “vascular access device” can be a medical device for vascular access including, but not limited to, a catheter such as a peripherally inserted central catheter (“PICC”), a central venous catheter (“CVC”), a midline catheter, an intravenous line such as a peripheral intravenous line (“Ply”), or the like.

Unless defined otherwise, all technical and scientific terms used herein have the same meaning as commonly understood by those of ordinary skill in the art.

Again, current technologies work to optimize placement of a user-selected medical device for vascular access after ultrasound imaging or the like; however, the user-selected medical device is not always the best medical device for vascular access in view of multifaceted considerations of applicable criteria. That, and time can be saved by at least narrowing a selection of medical devices before even performing the ultrasound imaging.

Disclosed herein are systems and methods for automatic determination of one or more medical devices for vascular access prior to direct patient assessment, which ensure best procedural outcomes starting with medical-device selection. Notably, such systems can incorporate automated data inputs, clinician-provided data inputs, or a combination thereof in decision-making processes to instantly provide clinicians recommendations for appropriate medical devices for vascular access to their patients. For example, a system can include a console and a display screen optionally integrated into the console. The console can include one or more processors and memory. The memory can include instructions configured to instantiate one or more processes when executed by the one-or-more processors for the automatic determination of the one-or-more medical devices for vascular access in accordance with a plurality of data inputs, various operating parameters, historical data, or a combination thereof. The automatic determination of the one-or-more medical devices can use at least logic, algorithms, machine learning including a machine-learning model trained with the historical data, artificial intelligence, or a combination thereof. The display screen can be configured to display the one-or-more medical devices recommended for vascular access.

### Systems

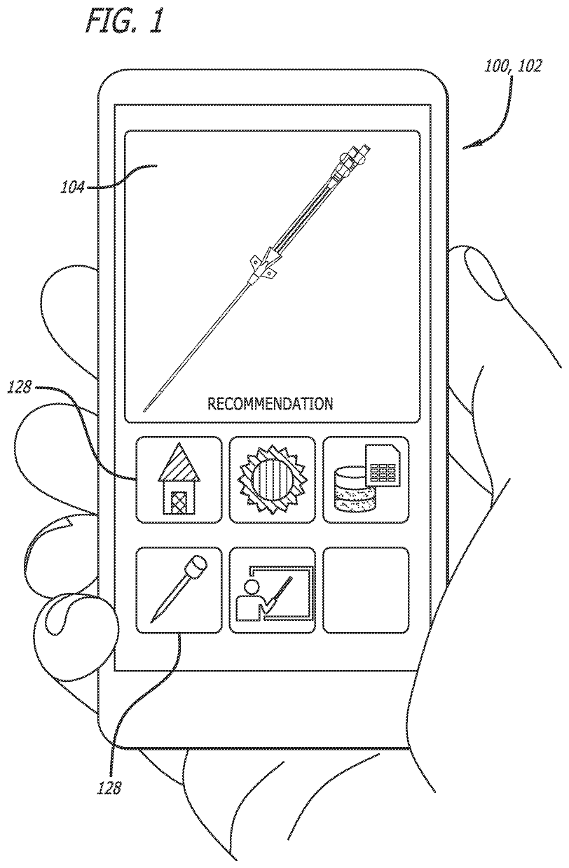

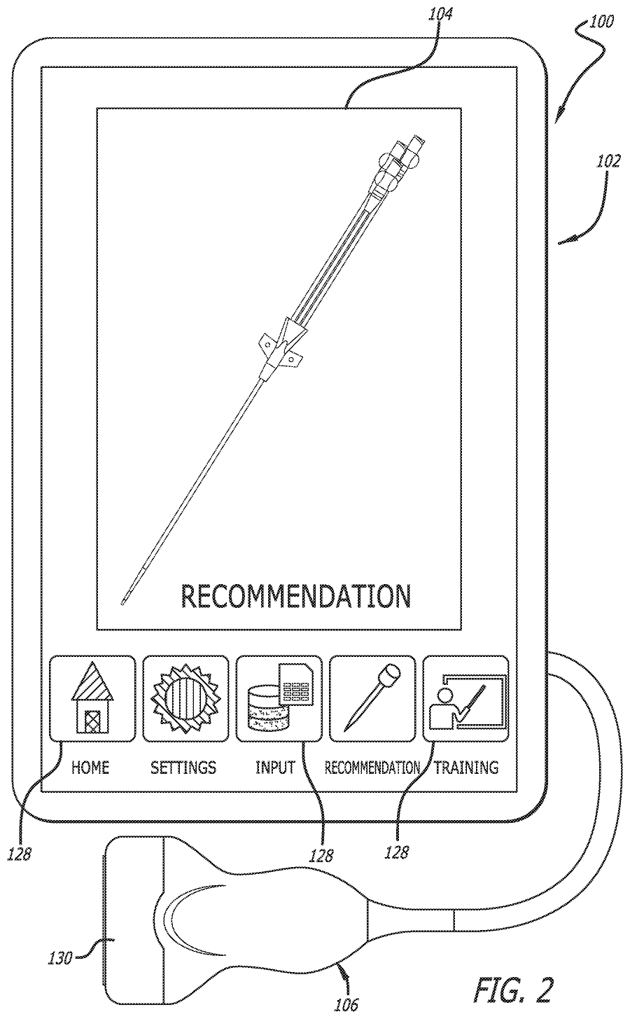

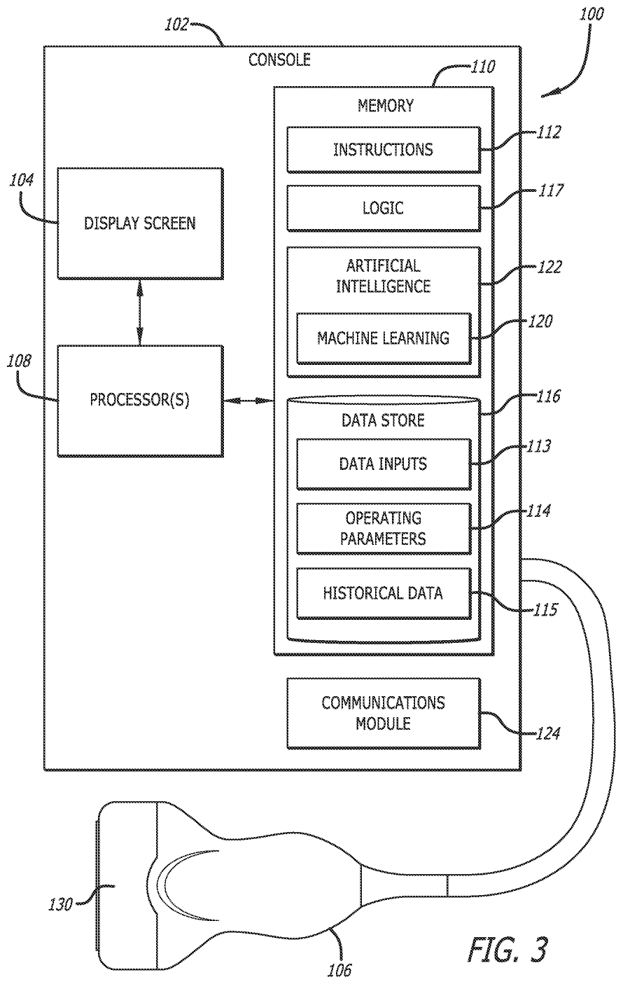

FIGS. 1 and 2 respectively illustrate a system 100 for automatic determination of one or more medical devices for vascular access prior to direct patient assessment in accordance with some embodiments. FIG. 3 illustrates a block diagram of the system 100 of FIG. 1 or 2 in accordance with some embodiments.

As shown, the system 100 can include a console 102 and a display screen 104 (e.g., a touchscreen) optionally integrated into the console 102. Notably, the system 100 shown in FIG. 1 is a portable computing device (e.g., smartphone, tablet computer, etc.) with the display screen 104 integrated therein, whereas the system 100 shown in FIG. 2, which might be located at a nursing station, vascular access-team station, or the like, is a computing device such as a desktop computer, desktop-replacement computer, special-purpose computer, or the like with the display screen 104 integrated therein, as shown, or in a standalone monitor. Further, the system 100 can also include an ultrasound probe 106 configured to operably couple with the console 102 in a wired or wireless connection for ultrasound imaging, a tip-location sensor, which is configured to rest upon a chest of a patient for locating a tip of a medical device (e.g., a catheter) within a vasculature of the patient while placing the medical device, a fiber-optic stylet for fiber-optic shape sensing, an infusion pump configured for infusing an infusate upon placement of the medical device, or a combination thereof. Notably, a tip-location sensor is shown and described in US 2020/0060643, which is incorporated herein in its entirety.

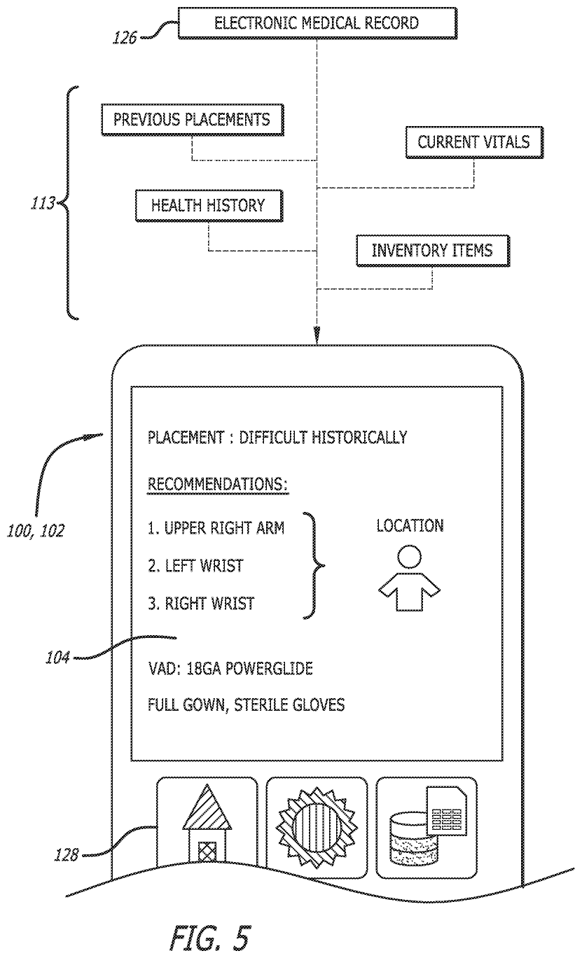

While the system 100 is configured for the automatic determination of the one-or-more medical devices for vascular access prior to the direct patient assessment, the system 100 can be further configured for other recommendations as well as reports. In an example, the system 100 can be further configured for automatic determination and recommendation of an insertion site for the vascular access with the one-or-more medical devices prior to the direct patient assessment. (See, for example, recommendations in FIG. 5.) In another example, the system 100 can be further configured for automatic determination and recommendation of additional procedure-related items for the vascular access with the one-or-more medical devices prior to the direct patient assessment. Such additional procedure-related items can include gowns, drapes, gloves based upon size, or a combination thereof. In yet another example, the system 100 can be further configured for automatically compiling and analyzing data pulled into the system 100 or manually input into the system 100 by a clinician to identify data trends for reporting internally to a clinic including the system 100 or externally to another care provider.

The console 102 can include one or more processors 108 and memory 110.

The memory 110 can include random-access memory (“RAM”) or non-volatile memory (e.g., electrically erasable programmable read-only memory [“EEPROM”]), and the one-or-more processors 108 and the memory 110 of the console 102 can be configured to control various functions of the system 100, as well as executing various operations (e.g., processing electrical signals from the ultrasonic transducers of the ultrasound probe 106, if present, into ultrasound images) during operation of the system 100 in accordance with executable instructions 112 therefor stored in the memory 110 for execution by the one-or-more processors 108. Indeed, the instructions 112 are configured to instantiate one or more processes when executed by the one-or-more processors 108 for the automatic determination of the one-or-more medical devices for vascular access prior to the direct patient assessment. The automatic determination of the one-or-more medical devices for vascular access is in accordance with a plurality of data inputs 113, various operating parameters 114, historical data 115, or a combination thereof stored at least temporarily (e.g., prior to a procedure) in a data store 116 for the automatic determination by at least logic 117, algorithms 118, machine learning 120, artificial intelligence 122, or a combination thereof of the console 102. The artificial intelligence 122 (e.g., an artificial neural network [“ANN”]) is trained via the machine learning 120 using known, acceptable medical-device recommendations for the plurality of data inputs 113 or the various operating parameters 114. Though, the automatic determination of the one-or-more medical devices for vascular access can be further in accordance with a plurality of patient condition parameters input by a measuring device (e.g., thermometer, blood-pressure monitor, blood-oxygenation monitor, etc.) configured to be operably coupled to the console 102, optionally, wired or wirelessly through a communications module 124. The plurality of patient condition parameters can include temperature, blood pressure, blood oxygenation, pH, lactate concentration, glucose levels, or a combination thereof.

The plurality of data inputs 113 can be automatically pulled into the system 100 or manually input into the system 100 by a clinician using the system 100.

As to the plurality of data inputs 113 being automatically pulled into the system 100, the plurality of data inputs 113 can be automatically pulled into the system 100 by way of the communications module 124 for communicating with an electronic medical-care-facility system, which, in turn, optionally includes access to patient data via electronic medical records 126 for patients. The electronic medical-care-facility system or the electronic medical records 126 for the patients can include orders for specific medical devices, specific medications (e.g., infusates), or a combination thereof for use by the logic 117, the algorithms 118, the machine learning 120, the artificial intelligence 122, or the combination thereof for the automatic determination of the one-or-more medical devices for vascular access. In addition, the electronic medical-care-facility system or the electronic medical records 126 for the patients can include patient data such as patient location, diagnostic data, or imaging data such as digital intravenous angiography (“DIVA”) for use by the logic 117, the algorithms 118, the machine learning 120, the artificial intelligence 122, or the combination thereof for the automatic determination of the medical device for vascular access.

As to manual input of the plurality of data inputs 113 into the system 100, the plurality of data inputs 113 can be manually input into the system 100 by a clinician using the system 100. For example, the plurality of data inputs 113 can include procedure type, one or more clinical rules, optionally, preloaded based upon common clinical standards for factoring into any automatic recommendations of the one-or-more medical devices for vascular access, clinician experience, one or more clinician preferences, inventory items such as medical-device inventory (see “inventory items” in FIG. 5), one or more orders for specific medical devices, patient data including patient condition (e.g., “health history” or “current vitals” as show in FIG. 5) or patient location (e.g., patient room number), emergent indication, prior ease or difficulty in blood-vessel access (e.g., “previous placements” as shown in FIG. 5), one or more orders for specific medications (e.g., infusates), one or more infusion-therapy parameters, one or more imaging parameters if the system 100 includes an imaging modality such as ultrasound imaging, dwell time, or a combination thereof. The procedure type can include at least an intention for blood aspiration. For each medical device of the one-or-more medical devices, the one-or-more clinical rules can include a purchase length for the medical device, blood-vessel occupancy of the medical device, dwell time for the medical device, or a combination thereof when the medical device is at least a catheter. The clinician experience can include clinician training for placing the one-or-more medical devices for vascular access, clinician proficiency for placing the one-or-more medical devices for vascular access, or both. The patient condition can include blood pressure, hydration, nutrition, temperature, or a combination thereof. The prior difficulty in blood-vessel access can include an indication of whether a blood vessel was visible, palpable, torturous, valvular, or a combination thereof during one or more previous clinic visits. The one-or-more infusion-therapy parameters can include fluid replacement, potassium replacement, heparin, insulin, one or more antibiotics, one or more vesicants, one or more irritants, blood, one or more blood products, pain medication, power-injection parameters, or a combination thereof. The one-or-more imaging parameters can include at least an intention for power injection while imaging in embodiments of the system 100 including the ultrasound probe 106.

Notwithstanding the demarcation set forth above between the plurality of data inputs 113 being automatically pulled into the system 100 or manually input into the system 100 by a clinician, it should be understood that some of the plurality of data described as being automatically pulled into the system 100 can alternatively be manually input into the system 100 by a clinician. Likewise, some of the plurality of data described as being manually input into the system 100 by a clinician can alternatively be automatically pulled into the system 100.

The various operating parameters 114, like the plurality of data inputs 113 set forth above, can be automatically pulled into the system 100 or manually input into the system 100 by a clinician using the system 100. The various operating parameters 114 can include confirmation of clinician training via completion of one or more on-board training modules. Clinicians having the confirmation of the clinician training can be allowed to use the system 100 for the automatic determination of the one-or-more medical devices for vascular access, whereas those not having the confirmation of the clinician training can be restricted from using the system 100 for the automatic determination of the one-or-more medical devices for vascular access. As set forth below, the display screen 104 can be configured to display the clinician messaging. Such clinician messaging can include the confirmation of the clinician training or recommendations for escalation. The escalation can include one or more alternative clinicians allowed to use the system 100 for the automatic determination of the one-or-more medical devices for vascular access in view of the one-or-more alternative clinicians having the confirmation of the clinician training. Notably, the clinician messaging can include the recommendations for the one-or-more alternative clinicians allowed to use the system 100, which can be further in view of the clinician proficiency.

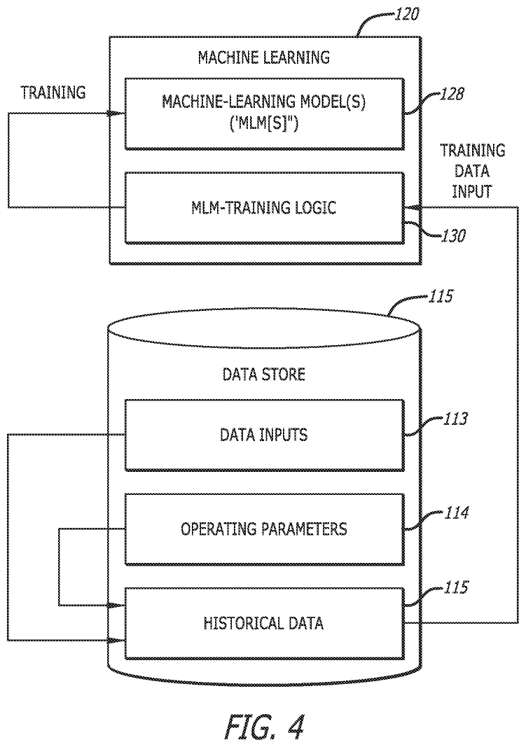

FIG. 4 illustrates training one or more MLMs 128 with the historical data 115 of the system 100 in accordance with some embodiments.

Adverting to the machine learning 120, the machine learning 120 can include the one-or-more MLMs 128 and MLM-training logic 130 as shown in FIG. 4. The MLM-training logic 130 can be configured to provide the one-or-more MLMs 128 with the historical data 115 as training data when training the one-or-more MLMs 128 to learn from the training data in accordance with supervised learning, semi-supervised learning, or unsupervised learning. Notably, the historical data 115 can include any of the plurality of data inputs 113, the various operating parameters 114, or a combination thereof previously automatically pulled into the system 100 or manually input into the system 100 by a clinician using the system 100. Such historical data 115 can also be labeled by the MLM-training logic 130 as appropriate by genus or species of data for the supervised or semi-supervised training. For example, a genus of data can include medical-device data or catheter data as a subgenus thereof, and species of data can include various makes and models of the medical devices or catheters of the medical-device data or catheter data.

As set forth below, the display screen 104 can be configured to display the one-or-more medical devices recommended for vascular access, which the clinician can choose from based upon an optionally provided percent likelihood of success, confidence level, or both for each medical device of the one-or-more medical devices. Clinician interactions with the system 100 such as a clinician's choice of a particular medical device over any other medical devices of the one-or-more medical devices recommended for vascular access can be automatically pulled into the system 100 and incorporated into the historical data 115. The clinician interactions with the system 100 can also include a clinician's feedback on whether the chosen medical device of the one-or-more medical devices recommended for vascular access was appropriate, successful, or the like, if not automatically tracked by the system 100, itself via ultrasound imaging or the tip-location sensor, after placing the medical device. Notably, as to tracking by the system 100 by ultrasound imaging, the tip-location sensor, fiber-optic shape sensing, or the like, a tracking record of the tracking can also be incorporated into the historical data 115 for training the one-or-more MLMs 128. In addition, the ease or difficulty in blood-vessel access with the chosen medical device at a recommended or alternatively chosen insertion site can be incorporated into the historical data 115, optionally, relationally, for subsequent use as the prior ease or difficulty in blood-vessel access of the plurality of data inputs 113. Such historical data 115 can, like that set forth above, be provided to the one-or-more MLMs 128 as training data when training the one-or-more MLMs 128 to learn from the training data. In this way, the one-or-more MLMs 128 can be continuously updated and improved for the automatic determination of the one-or-more medical devices for vascular access.

The display screen 104 can be integrated into the console 102, as shown, or the display screen 104 can be part of a standalone monitor configured to operably couple with the console 102 as set forth above. As shown in FIGS. 1 and 2, the display screen 104 can be configured to display the one-or-more medical devices recommended for vascular access, optionally, accompanied by a percent likelihood of success, a confidence level, or both for each medical device of the one-or-more medical devices, thereby ultimately allowing a clinician to choose the medical device of the one-or-more medical devices. When the system 100 includes the ultrasound probe 106, the display screen 104 can also be configured to display an ultrasound image including one or more blood vessels below a skin surface of a patient. Notably, the display screen 104 can also be configured to display one or more on-screen buttons 132 (e.g., a home button, a settings button, a data-input button, a medical-device recommendation button, a training button, etc.) enabling the clinician to interact with various aspects of the system 100. For example, the one-or-more on-screen buttons 132 can include the example medical-device recommendation button, which the clinician can press prior to any direct patient assessment for the automatic determination of the one-or-more medical devices for vascular access. In addition, the display screen 104 can be further configured to display clinician messaging such as the confirmation of the clinician training or the one-or-more alternative clinicians allowed to use the system 100 set forth above.

Notably, the system 100 need not include the display screen 104 to display the one-or-more medical devices recommended for vascular access. Indeed, whether the display screen 104 is present or not, the system 100 can be configured for physical delivery of the one-or-more medical devices recommended for vascular access or even automatic placement of the one-or-more medical devices recommended for vascular access such as in an autonomous vascular-access-device placement kiosk.

While not shown, the console 102 can further include a power connection configured to enable an operable connection to an external power supply. An internal power supply (e.g., a battery) can also be employed either with or exclusive of the external power supply. Power management circuitry of the console 102 can regulate power use and distribution.

When present, the ultrasound probe 106 can include a probe head 134 housing an array of ultrasonic transducers, wherein the ultrasonic transducers are piezoelectric ultrasonic transducers or capacitive micromachined ultrasonic transducers (“CMUTs”). The probe head 134 is configured for placement against a skin surface of a patient proximate a prospective site for placing a medical device for vascular access, where the ultrasonic transducers in the probe head 134 can generate ultrasound signals and emit the generated ultrasound signals into the patient in a number of pulses, receive reflected ultrasound signals or ultrasound echoes from the patient by way of reflection of the generated ultrasonic pulses by a body of the patient, and convert the reflected ultrasound signals into corresponding electrical signals for processing into the ultrasound image by the console 102.

### Methods

Methods can include a method of the system 100 for automatic determination of the one-or-more medical devices for vascular access prior to the direct patient assessment. Such a method can include one or more steps selected from at least an instantiating step, a device-recommending step, and a displaying step.

The instantiating step can include executing the instructions 112 in the memory 110 of the console 102 by the one-or-more processors 108 of the console 102, thereby instantiating the one-or-more processes for the automatic determination of the one-or-more medical devices for vascular access in accordance with the plurality of data inputs 113, various the various operating parameters 114, or the combination thereof. The instantiating step can be initiated by a clinician simply powering up the system 100, particularly if the system 100 includes the special-purpose computer set forth above.

The device-recommending step can include determining and, subsequently, recommending the one-or-more medical devices recommended for vascular access, optionally, accompanied by a percent likelihood of success, a confidence level, or both for each medical device of the one-or-more medical devices. The device-recommending step can be different than the displaying step, as the device-recommending step can be data processing with the logic 117, the algorithms 118, the machine learning 120, the artificial intelligence 122, or the combination thereof in preparation for the displaying step.

The displaying step can include displaying on the display screen 104 optionally integrated into the console 102 the one-or-more medical devices recommended for vascular access.

Notably, the system 100 set forth above is configured to function in a number of different ways and, thereby, perform a number of additional steps to the instantiating step, the device-recommending step, and the displaying step. The number of additional steps gleaned by way of the number of different ways the system 100 functions are incorporated into this section so as to not burden the specification. In an example, the system 100 can be further configured as set forth above for the automatic determination of the additional procedure-related items for the vascular access with the one-or-more medical devices prior to the direct patient assessment. As such, the method can further include a procedure-related-items recommending step that includes determining and recommending additional procedure-related items for the vascular access with the one-or-more medical devices prior to the direct patient assessment. In another example, the system 100 can be further configured as set forth above for the automatic compiling and analyzing of the data pulled into the system 100 or manually input into the system 100 by a clinician. As such, the method can further include a compiling or analyzing step that respectively includes automatically compiling or analyzing the data pulled into the system 100 or manually input into the system 100 by the clinician. Because such compiling and analyzing of the data is for identifying data trends to report, the method can further include a data-reporting step that includes reporting internally to a clinic having the system 100 or externally to another care provider, and so on.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures can be made from the particular embodiments disclosed herein without departing from the scope of the concepts provided herein.

What is claimed is:

1. A system for automatic determination of one or more medical devices for vascular access prior to direct assessment of a patient, comprising:
a console including:
one or more processors;
memory including instructions configured to instantiate one or more processes when executed by the one-or-more processors for the automatic determination of the one-or-more medical devices for vascular access in accordance with a plurality of data inputs, various operating parameters, historical data of the plurality of data inputs and various operating parameters, or a combination thereof, the automatic determination of the one-or-more medical devices using at least logic, algorithms, machine learning including a machine-learning model trained with the historical data, artificial intelligence, or a combination thereof.

2. The system of claim 1, wherein the plurality of data inputs are automatically pulled into the system.

3. The system of claim 2, wherein the plurality of data inputs include patient condition parameters measured by a measuring device, optionally, operably connected to the system, the patient condition parameters including temperature, blood pressure, blood oxygenation, pH, lactate concentration, glucose levels, or a combination thereof.

4. The system of claim 2, wherein the plurality of data inputs include orders or patient data from an electronic medical-care-facility system, which, in turn, optionally includes access to the patient data via electronic medical records for patients.

5. The system of claim 4, wherein the orders are for specific medical devices, specific medications, or a combination thereof.

6. The system of claim 1, further comprising:
a display screen optionally integrated into the console, the display screen configured to display the one-or-more medical devices recommended for vascular access.

7. The system of claim 6, wherein the various operating parameters include confirmation of clinician training via completion of one or more on-board training modules, the display screen further configured to display clinician messaging with the confirmation of the clinician training.

8. The system of claim 7, wherein only clinicians having the confirmation of the clinician training are allowed to use the system for the automatic determination of the one-or-more medical devices for vascular access.

9. The system of claim 8, wherein the display screen is further configured to display the clinician messaging with recommendations for escalation including one or more alternative clinicians allowed to use the system for the automatic determination of the one-or-more medical devices for vascular access in view of the one-or-more alternative clinicians having the confirmation of the clinician training.

10. The system of claim 1, wherein the plurality of data inputs are manually input into the system by a clinician using the system.

11. The system of claim 10, wherein the plurality of data inputs include procedure type, one or more clinical rules, clinician experience, one or more clinician preferences, medical-device inventory, one or more orders for specific medical devices, patient data including patient condition or patient location, emergent indication, prior difficulty in blood-vessel access, one or more orders for specific medications, one or more infusion-therapy parameters, one or more imaging parameters if the system includes an imaging modality, dwell time, or a combination thereof.

12. The system of claim 11, wherein the one-or-more clinical rules include purchase length for a medical device, blood-vessel occupancy of the medical device, dwell time for the medical device, or a combination thereof when the medical device is a catheter.

13. The system of claim 11, wherein the one-or-more clinical rules are factored into any automatic recommendations of the one-or-more medical devices for vascular access.

14. The system of claim 11, wherein the one-or-more infusion-therapy parameters include fluid replacement, potassium replacement, heparin, insulin, one or more antibiotics, one or more vesicants, one or more irritants, blood, one or more blood products, pain medication, power-injection parameters, or a combination thereof.

15. The system of claim 11, wherein the one-or-more imaging parameters include at least an intention for power injection while imaging.

16. The system of claim 11, wherein the procedure type includes at least an intention for blood aspiration.

17. The system of claim 11, wherein the prior difficulty in blood-vessel access includes a determination of whether a blood vessel was visible, palpable, torturous, valvular, or a combination thereof.

18. The system of claim 11, wherein the patient condition includes blood pressure, hydration, nutrition, temperature, or a combination thereof.

19. The system of claim 11, wherein the clinician experience includes clinician training for placing the one-or-more medical devices for vascular access or clinician proficiency for placing the one-or-more medical devices for vascular access.

20. The system of claim 1, further comprising an ultrasound probe operably coupled to the console for ultrasound imaging.

21. The system of claim 20, further comprising a tip-location sensor configured to rest upon a chest of a patient, the tip-location sensor configured to locate a tip of a medical device within a vasculature of the patient.

22. The system of claim 20, further comprising an infusion pump.

23. The system of claim 1, wherein the console is a portable computing device with a display screen integrated therein.

24. The system of claim 1, wherein the system is further for automatic determination of additional procedure-related items for the vascular access with the one-or-more medical devices prior to the direct patient assessment patient, the additional procedure-related items including gowns, drapes, gloves based upon size, or a combination thereof.

25. The system of claim 1, wherein the automatic determination of the one-or-more medical devices for vascular access is accompanied by a percent likelihood of success, a confidence level, or both, thereby ultimately allowing a clinician to choose a medical device of the one-or-more medical devices.

26. The system of claim 1, wherein the one-or-more processes are further for automatically compiling and analyzing data to identify data trends for reporting internally to a clinic including the system or externally to another care provider.

27. The system of claim 1, wherein the system is configured to train the machine-learning model with the historical data including a clinician-chosen medical device among the one-or-more medical devices recommended for vascular access, clinician feedback on whether the clinician-chosen medical device was successful for vascular access, a tracking record for the clinician-chosen medical device, or a combination thereof.

28. A method of a system for automatic determination of one or more medical devices for vascular access prior to direct patient assessment, comprising:
executing instructions in memory of a console by one or more processors of the console to instantiate one or more processes for the automatic determination of the one-or-more medical devices for vascular access in accordance with a plurality of data inputs, various operating parameters, historical data, or a combination thereof, the automatic determination of the one-or-more medical devices using at least logic, algorithms, machine learning including a machine-learning model trained with the historical data, artificial intelligence, or a combination thereof.

29. The method of claim 28, further comprising displaying on a display screen optionally integrated into the console the one-or-more medical devices recommended for vascular access.
